(19) United States
(12) Patent Application Publication
(10) Pub. No.: US 2024/0261705 A1
(43) Pub. Date: Aug. 8, 2024

(54) CHROMATOGRAPHY MEDIUM FOR USE IN PURIFICATION OF ENVELOPED VIRUS PARTICLES OR EXOSOMES

(71) Applicant: Cytiva BioProcess R&D AB, Uppsala (SE)

(72) Inventors: Jean-Luc Maloisel, Uppsala (SE); Asa Hagner McWhirter, Uppsala (SE); Ola Lind, Uppsala (SE); Frederic Leroux, Uppsala (SE)

(21) Appl. No.: 18/566,551

(22) PCT Filed: Jun. 27, 2022
(86) PCT No.: PCT/EP2022/067633

§ 371 Date: Dec. 1, 2023

(30) Foreign Application Priority Data
Jul. 2, 2021 (GB) 2109610.2

(51) Int. Cl. B01D 15/36; B01J 41/20; C12N 7/02

(52) U.S. Cl. CPC B01D 15/363; B01J 41/20; C12N 7/02

(57) ABSTRACT

An anion exchange chromatography medium (1) for use in purification of enveloped virus particles or exosomes from a feed, the anion exchange chromatography medium comprising a support material being functionalized with a ligand comprising a diamine functionality generating at least one weak anion exchange group to an ionic capacity of 10-500 μmol/mL.

### TECHNICAL FIELD

The present disclosure relates to an anion exchange chromatography medium for use in purification of enveloped virus particles or exosomes from a feed. It is also an object to provide a process of purifying enveloped virus particles from a feed using this anion exchange chromatography medium.

### BACKGROUND ART

The lentivirus (LV) is classed as retrovirus, and it has a single stranded RNA genome with a reverse transcriptase enzyme. Lentiviruses consist of a viral envelope with glycosylated proteins acting as ligands that has affinity to host cells receptors in the outer cell membrane surface. The virus perform transcription of the viral genetic material upon entering the cell. The viral genome consists of RNA sequences that code for specific proteins that facilitate the incorporation of the viral sequences into the host cell genome.

The virus infects the host cells by docking onto the host cell surface CD 4 glycoproteins. Then the virus injects its material into the host cells cytoplasm were the reverse transcriptase enzyme generates reverse transcription of the viral RNA so a viral DNA genome is produced which is sent to the nucleus of the host cell were it is incorporated in the host cell genome. The host cell starts to transcribe the virus RNA and express viral proteins that forms the capsid. The LV RNA and the viral proteins then assemble and the newly formed virions leave the host cell when enough are made.

In gene therapy, the virus is modified to act as a vector to insert beneficial genes into cells. The benefit using LV as viral vector is that it can penetrate the nuclear envelope in dividing and non-dividing cells, unlike other retroviruses that only penetrate the cells while they are under mitosis. Many cell types in adult persons do not divide and LV might be the only option to transfer genetic material into the cell. Genetically modified LVs used in cell and gene therapy have proven to be promising candidate for curing diseases such as diabetes, prostate cancer, chronic granulomatous disease and vascular diseases. It is thus important to modify the virus genome so it cannot replicate itself and that it is incorporated permanently in the cells genome. The transduction of human cells by genetically modified lentiviruses is most made ex-vivo by transfection of human T-cells.

To produce a lentivirus, several plasmids are transfected into a so-called packaging cell line. One or more plasmids, generally referred to as packaging plasmids, encode the virion proteins, such as the capsid and the reverse transcriptase. Another plasmid contains the genetic material to be delivered by the vector. It is transcribed to produce the single-stranded RNA viral genome and is marked by the presence of the ψ (psi) sequence. This sequence is used to package the genome into the virion. To use lentivirus in gene therapy it is necessary to purify the virion from cell impurities like host cell proteins and DNA and excess plasmids after transfection. Normally, the harvested host cells producing lentivirus are nuclease treated and lentivirus is purified with several filtration techniques, such as normal flow microfiltration, ultrafiltration and dia-filtration, to reduce the level of impurities to approved levels.

Today's downstream purification processes of lentivirus is often synonymous with low recovery of infectious viruses since lentivirus is unstable and sensitive to shear forces, buffer components such as salt and degrades quickly in room temperature and time-consuming multistep processes is not beneficial. LV's are also only stable in a very narrow pH range, 7.0-7.4 (Kinetic Analyses of Stability of Simple and Complex Retroviral Vectors, F. Higashikawa et al., Virology 280, 124-131 (2001)), and conductivity window (<0.2M NaCl) (Process development of lentiviral vector expression, purification and formulation for gene therapy applications, Doctoral thesis, Sara Nilsson, U C L, 2016), of processing solutions which make the downstream purification process challenging to obtain high yields of infectious LV in the final formulation.

There is, hence, a need for improved or at least alternative methods of purifying lentivirus particles and also other encapsulated virus particles from feed.

### SUMMARY OF THE INVENTION

It is an object of the present disclosure to provide an anion exchange chromatography medium for use in purification of enveloped virus particles or exosomes. It is also an object to provide a process of purifying enveloped virus particles or exosomes from a feed using this anion exchange chromatography medium.

The invention is defined by the appended independent patent claims. Non-limiting embodiments emerge from the dependent patent claims, the appended drawings and the following description.

According to a first aspect there is provided an anion exchange chromatography medium for use in purification of enveloped virus particles or exosomes from a feed, the anion exchange chromatography medium comprising a support material being functionalized with a ligand comprising a diamine functionality generating at least one weak anion exchange group to an ionic capacity of 10-500 μmol/mL.

The anion exchange chromatography medium may be used for purification of enveloped virus particles or exosomes. Enveloped viruses are budding off from the host cells and have an outer lipid bilayer derived from the cell membrane containing viral glycoprotein. Inside the enveloped particle there is a protein capsid containing the viral genetic material. The envelope is critical for the infection of host cells (bind and fuse with host cell membrane) but is very sensitive to shear forces, salt, pH and detergents for example. Conditions during production and purification are important to retain infectivity of the virus and maximize the recovery of functional infectious virus. Examples of such enveloped viruses are DNA viruses, such as herpesvirus, oxvirus, hepadnavirus and asfarviridae; RNA viruses, such as flavivirus, alphavirus, togavirus, coronavirus, hepatitis D, orthomyxovirus, paramyxovirus, rhabdovirus, bunyavirus and filovirus; and retroviruses such as lentiviruses.

The support material is functionalized with ligands to an ionic capacity (number of charged functional groups per ml medium (μmol/ml)) of 10-500 μmol/mL, or in the range of 50-300 μmol/mL or 100-300 μmol/mL. No known affinity ligands exist for these kind of enveloped virus particles or exosomes. Development of affinity ligands for enveloped viruses is challenging due to a common requirement of harsh elution conditions that is likely to harm the virus. An ion exchange capture solution would enable milder elution conditions and would generate an improved process with higher recoveries.

The at least one weak anion exchange group may comprise multimodal weak anion-exchange groups, i.e. the anion-exchange group provides at least two different, but co-operative, sites which interact with the compound to be bound (i.e. the enveloped virus particle or exosome). For example, one of these sites may give an attractive type of charge-charge interaction between the ligand and the substance of interest. The other site may contribute to the binding by introducing a second local charge or by increasing the local amount of solvating water, which is impacting the binding capacity.

The weak anion exchange group may be positively charged or partially positively charged at a pH of 6-10.

Such positively charged or partially positively charged weak anion exchange group may attract an enveloped virus particle or exosome being negatively charged at neutral pH, such as lentivirus particles.

The weak anion exchange group may be positively charged or partially positively charged at a pH of 6-10 or, 6-9.5, 6-9 or 6-8.

Weak ionic exchange groups means that there is a gradient according to the pH from fully charged to not charged, having at PI a neutral charge (same amount of + and −). Strong anionic exchange groups, based on a quaternary amine, are on the contrary always charged. Almost all other anionic exchange groups, not based on a quaternary amine, are weak, i.e. the charge varies (and can be zero) within a reasonable range of pH used (such as e.g. pH 2-11).

The ligand or a portion of the ligand may be described by the formula:

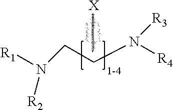

wherein X is selected from H, OH or a C₁-3 group, and
R1, R2, R3 and R4 are independently selected from H, and a C₁₋₃ group,
wherein a C₃ group is straight or branched,
wherein a C₁₋₃ group comprises groups independently selected from OH, O—C₁₋₂, S—C₁₋₂, NH, NHR, NR₂,
wherein R is selected from H and a C₁₋₃ group.

The ligand or a portion of the ligand may be described by the formula above. This means that the diamine ligand can constitute part of a larger structure such as a polymer.

The ligand or a portion of the ligand can be part of larger structures generated for example by the reaction of the solid support with lower molecular weight amine chemical containing leaving groups such as 2-Chloro-N,N-diethylethylamine (DEAE), 2-Chloro-N,N-diethylethylamine, 2-Chloroethylamine, 3-Chloropropylamine, 2-Chloro-N,N-dimethylethylamine, 3-Chloro-N-methylpropan-1-amine.

In the ligand or the portion of the ligand comprising a diamine functionality generating at least one weak anion exchange group described by the formula above, the two amines may be separated by 2-4 carbon atoms, each amine group may be substituted by two R groups, which may be chosen from H and alkyl groups C₁₋₄ and that can be branched and/or also substituted by other groups such as hydroxyl, amines, ether and thio ether, these later being restricted to 3-8 atoms.

The ligand described by the formula above may be selected from N,N,N′-triethylethylenediamine, diethylenetriamine, N,N′-dimethylethylenediamine, N-methylethylenediamine, 1,3-diaminopropane, 1,3-diamino-2-hydroxypropane, 2-methyl-1,3-propanediamine and N,N-diethylethylenediamine.

The support material may be selected from monoliths, membranes, porous beads, non-porous beads, magnetic beads, or expanded bed media.

Beads of different bead sizes may be used, such as beads having a diameter of 1-120 μm, or 10-120 μm. The beads may for example be of agarose.

Monoliths are single pieces of porous materials characterized by a highly interconnected network of channels with diameters in the range of 10-4000 nm.

Membrane materials may be inorganic-organic (e.g., an alkoxysilane coated on glass fiber), alumina membranes and organic materials (i.e., cellulose and its derivatives, regenerated cellulose, nylon, polyethersulfone, polypropylene, polyvinylidene, etc).

Support material may be a woven material.

The support material may be a non-woven fibrous material having an effective pore size of 0.1-2.0 μm.

The support material may be a non-woven material, for example comprising fibers, such as from cellulose, having an effective pore size of 0.1-2.0. The chromatography support material could comprise a convection-based chromatography matrix. Said convection-based chromatography matrix may be a fibrous substrate. Said fibrous substrate may be based on electrospun polymeric fibers or cellulose fibers, optionally non-woven fibers. The fibrous substrate may thus be a fibrous non-woven polymer matrix. The fibers comprised in said fibrous substrate have a cross-sectional diameter of 10-1000 nm, such as 200-800 nm, 200-400 nm or 300-400 nm. Such a fibrous substrate can be found in a HiTrap Fibro unit from Cytiva.

The ligand may be connected to the support material through an extender group selected from polysaccharide structures and polymeric structures.

The extender group may be e.g. dextrane, acrylamides or polyglycidol. If dextran is used as extender it may be in a range of molecular weight between 5 000 to 2M Dalton. The extender group used depends on the support material and which ligand to immobilize/connect to the support material.

According to a second aspect, there is provided an anion exchange chromatography comprising the anion exchange chromatography medium described above.

According to a third aspect, there is provided a process of purifying enveloped virus particles or exosomes from a feed, the process comprising to obtain a solution comprising the enveloped virus particles and one or more impurities, adding the solution to the anion exchange chromatography medium described above at a pH of 6-8, eluting the encapsulated virus particles or exosomes from the anion exchange chromatography medium by contacting the anion exchange chromatography medium with an elution buffer having a salt concentration of at most 0.65 M, and thereafter collecting the thus formed eluate containing enveloped virus particles or exosomes.

The feed comprising the enveloped virus particles or exosomes may be produced by a cell line such as for example HEK 293 cells (human embryo kidney). The feed may have been pre-treated, clarified, before being applied to the anion exchange chromatography medium. In such a clarified harvest the amount of solids has been reduced and the one or more impurities may be soluble impurities such as host cell proteins and DNA.

The elution buffer may have a salt concentration of at most 0.65 M. This relatively low salt concentration is used as enveloped viruses and exosomes have a reduced stability at higher conductivities, and especially so at concentrations higher than 0.65 M. In one embodiment the salt concentration is at most 0.45 M.

In the elution step, direct dilution to decrease conductivity in a buffer containing sucrose could stabilize the virus and thereby improve the recovery.

A stabilizer such as sucrose may be added to improve stability of the enveloped virus particles or exosomes. The stabilizer may be added in all mobile phases and in formulation solutions.

Typically, elution of enveloped viruses or exosomes from the anion exchange chromatography medium having a support material being a convection-based fibrous substrate, such as a fibrous non-woven polymer matrix, can be made at flowrates down to a few seconds residence time, i.e. 60 MV/min, and if required up to several minutes residence time (max 6 min) 0.2 MV/min. Optimal residence time for such fibrous support materials is 5-20 MV/min. For resins, typical residence times are 1-8 min and a sufficient residence time is often obtained after 4 min.

The process described above may further comprise, after the eluting step, a step of adding the eluate to a multimodal chromatography resin.

When eluting the target virus particle from the anion exchanger, there will still be residual impurities in the eluate. By applying a multimodal chromatograph column in-line after the anion exchanger, residual impurities may be adsorbed. In one example a Capto Core 700 can be used for adsorbing impurities under 700 kDa. Other multimodal chromatography resins may be used with other cut-offs, such as 400 kDa or 1000 kDa. The flow rate should be lowered in this step to allow the impurities to diffuse into the resin pores.

### BRIEF DESCRIPTION OF THE DRAWINGS

FIG. 1 shows an anion exchange chromatograph for encapsulated virus particle or exosome purification.

FIG. 2 schematically illustrates a process of purifying enveloped virus particles or exosomes from a feed. The process comprises to add a solution comprising the enveloped virus particles or exosomes and one or more impurities to the anion exchange chromatography medium shown in FIG. 1.

FIGS. 3a-3d shows a support material for an anion exchange chromatography medium, a fibrous non-woven polymer material of cellulose, Fibro, functionalized in four different ways.

FIG. 4a illustrates a reaction scheme of preparing an anion exchange chromatography medium with tertiary amine groups in which a non-woven polymer matrix comprising cross-linked fibers with hydroxyl reactive groups is reacted with 2-chloro-n,n-diethylamine hydrochloride to form a functionalized matrix with diethylaminoethyl groups.

FIG. 4b illustrates a reaction scheme of preparing an anion exchange chromatography medium with tertiary amine groups in which a non-woven polymer matrix comprising fibers with vinylsulfone reactive groups is reacted with N,N-diethylethylenediamine to form a functionalized matrix with N,N-diethylethylenediamine groups.

FIG. 4c illustrates a reaction scheme of preparing an anion exchange chromatography medium with tertiary amine groups in which magnetite agarose particles first are functionalized with dextran and thereafter hydroxyl reactive groups on the dextran are reacted with 2-chloro-n,n-diethylamine hydrochloride to form functionalized particles with diethylaminoethyl groups.

FIG. 5a shows capture of lentivirus using Fibro DEAE IC193 as the anion exchange chromatograpy medium using step elutions with increasing salt concentration. (Striped pattern show infections recovery and filled black bars show total virus particles (p24 ELISA)).

5b shows capture of lentivirus using Fibro DEAE IC207 as the anion exchange chromatograpy medium using step elutions with increasing salt concentration. (Striped pattern show infections recovery and filled black bars show total virus particles (p24 ELISA)).

FIG. 5c shows the reproducibility of lentivirus capturing using the anion exchange chromatography materials of FIG. 5a and FIG. 5b, respectively.

### DETAILED DESCRIPTION

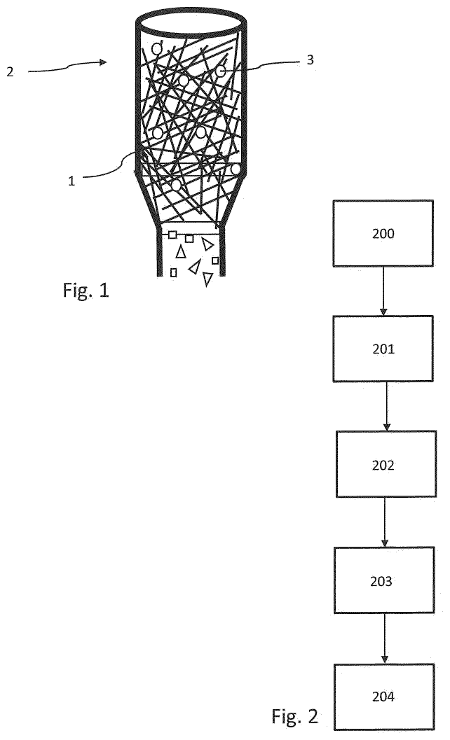

FIG. 1 shows an anion exchange chromatography 2 for purification of encapsulated virus particles or exosomes 3 from a feed. The anion exchange chromatography medium 1 comprises a support material being functionalized with a ligand comprising a diamine functionality generating at least one weak anion exchange group to an ionic capacity of 10-500 μmol/mL, or in the range of 50-300 μmol/mL or 100-300 μmol/mL. The weak anion exchange group may be positively charged or partially positively charged at a pH of 6-10. The invention uses a method for determining the dynamic small ion binding capacity (DBC) of Fibro membranes functionalized with AIEX ligands, such as DEAE, DAX and Q ligands but should be generally applicable for any AIEX ligand. 25 mm diameter membrane discs were attached in a membrane holder device suited to allow chromatography of the membranes, and run on an ÄKTA Explorer 10 system equipped with sample pump.

The method is essentially a conductometric titration where added HCl protonates deprotonated weak AIEX ligands or neutralizes and displaces OH bound to strong AIEX ligands. In contrast to protein DBC methods, the conductivity signal, rather than a UV signal, of the permeate is monitored in the ÄKTA system. The method consists of the following steps:
1. Rinsing of ÄKTA system and membrane (loaded in PEEK device).
2. Loading of membrane with excess NaOH
3. Rinsing of membrane with MQ water
4. Rinsing HCl solution in bypass
5. Loading membrane with HCl and monitor conductivity breakthrough
6. Reloading membrane with excess NaOH (optional, prep for normalization by weight)
7. Rinsing of membrane with MQ water

Each membrane batch is analyzed with triplicate discs. Normalization is done by disc volume and optionally also through disc dry weight.

The support material may be selected from monoliths, membranes, porous beads, non-porous beads, magnetic beads, or expanded bed media.

In FIG. 2 is schematically illustrated a process of purifying enveloped virus particles or exosomes from a feed. The process comprises to obtain 200 a solution, a feed, comprising the enveloped virus particles or exosomes and one or more impurities. The solution is added 201 to the anion exchange chromatography medium 1 at a pH of 6-10, and thereafter the encapsulated virus particles or exosomes are eluted 202 from the anion exchange chromatography medium 1 by contacting the anion exchange chromatography medium 1 with an elution buffer having a salt concentration of at most 0.65 M, and collecting 204 the thus formed eluate containing enveloped virus particles or exosomes. After the eluting step 202, the eluate may be added 203 to a multimodal chromatography resin to remove any residual impurity.

In the experimental section below specific examples of the method and functionalized support material are shown and discussed when purifying lentivirus particles.

### EXPERIMENTAL

### Lentivirus Feed Material

Clarified LVV GFP (Lentiviral vector encoding green fluorescent protein) (stored in −80° C. freezer in 40 mL aliquots), was produced in a 3 L Bioflo bioreactor, LVV was produced in HEK 293 cells, benzonase treated and normal flow filtrated and clarified by a filter train with a smallest cut-off of 0.2 μm.

### Support Material

The support material used was a fibrous non-woven polymer material of cellulose (hereinafter called Fibro) prepared by laminating 10 layers of electro-spun fiber layers into a sheet. Magnetite agarose (sepharose) beads were also used as support material.

### Fibro

### Preparation of Glycidol Vinylsulfone Cellulose Membrane (Fibro-VS)

50 laser-cut cellulose acetate discs of 32 mm diameter were placed between two fine polypropylene gauzes of dimension 900 mm by 95 mm. Ethanol was sprayed on the gauze so as to fully wet the discs. The gauzes were slowly wrapped around a hollow cylindrical core of 60 mm diameter and secured in place. The entire core was placed in a beaker and the discs were washed with distilled water (4×600 ml). The wash solution was removed and replaced with 350 ml 0.5M KOH solution. The discs were treated with the KOH solution for 10 mins with stirring, before the addition of 100 ml glycidol. The reaction media was stirred vigorously over the discs for 2 hours. After this time, the supernatant liquid was removed and the discs held between the gauzes were washed with distilled water (4×600 ml) to give a clean intermediate that was used without further modification for the next step.

Thereafter, 25 discs were taken from the glydicol step and setup in the same way described above. The new core was placed in 500 ml H₂O, which contained 37.5 g Na₂CO₃ and 150 ml MeCN. The mixture was stirred vigorously while 100 ml divinylsulfone was added dropwise over 60 minutes. The reaction mixture was then stirred vigorously for 16 hours. After this time, the supernatant liquid was decanted and the discs held between gauzes were washed with 600 ml acetone:H₂O (1:1) 3 times then with distilled H₂O (1×600 ml). The clean intermediate was used for the next step without further modification.

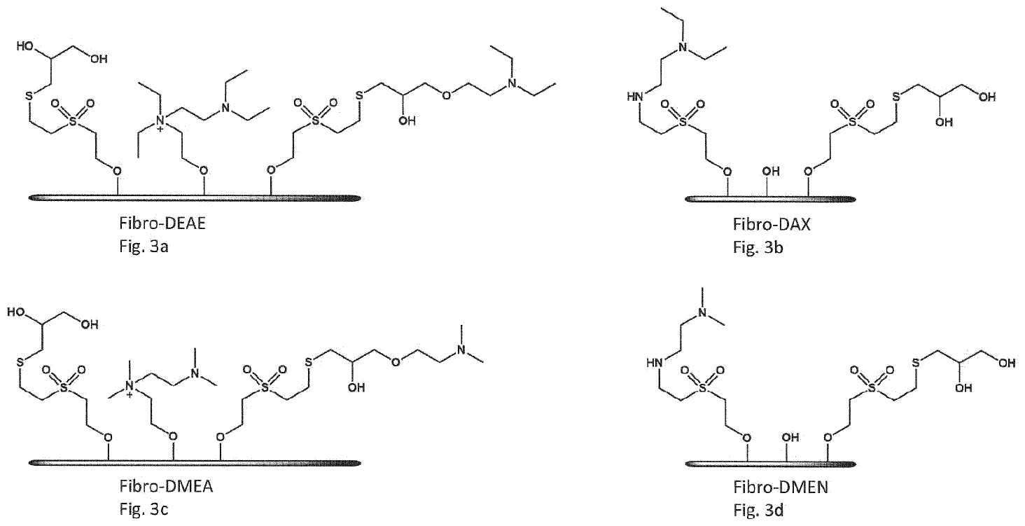

### DEAE (DiEthylAminoEthyl) Functionalization of Fibro (FIG. 3a)

The following protocols were used for the DEAE functionalization:

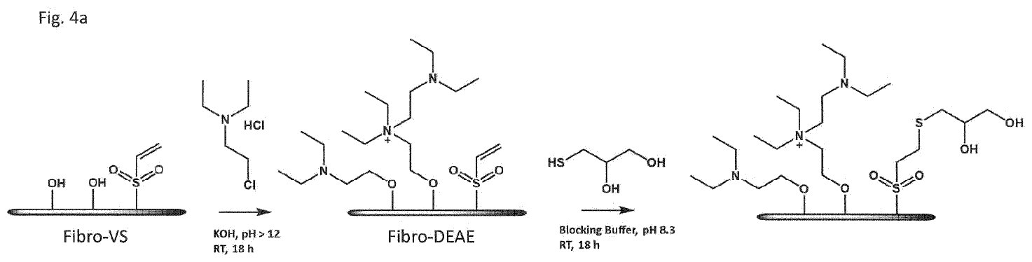

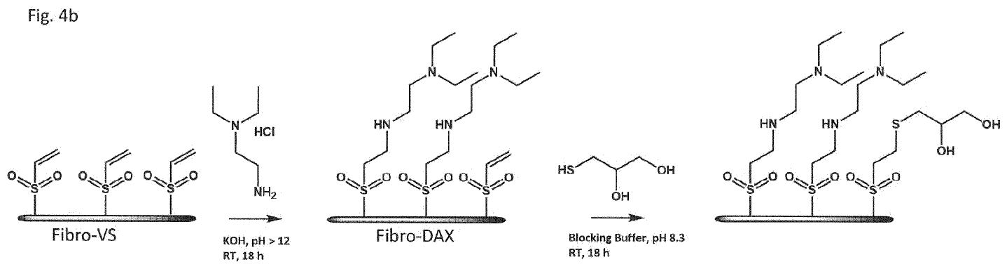

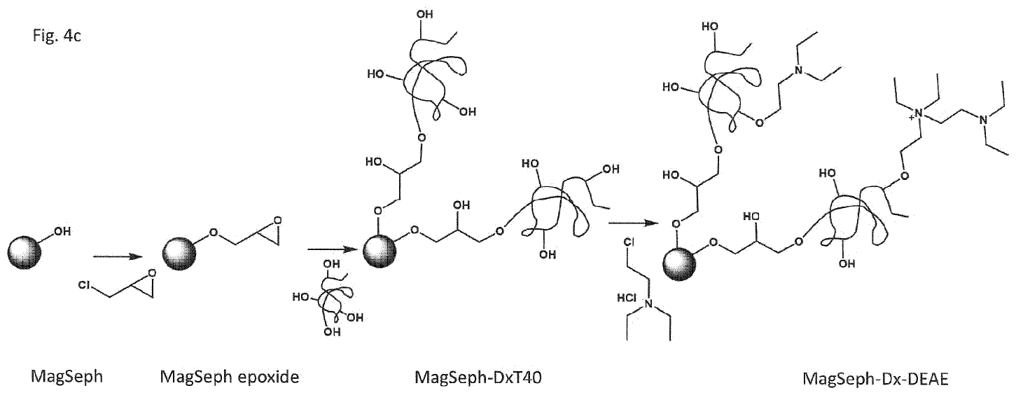

### DEAE Coupling Before Vinylsulfone Deactivation (See FIG. 4a)

20 discs Fibro-VS from above were washed with 150 ml DV20, 4 times in a polypropylene (PP) container. Following this, 2 g KOH was dissolved in 25 ml deionised water and added to the Fibro-VS discs for 30 minutes. Thereafter, 1.9 ml of 2-(Diethylamino)ethylchloride Hydrochloride (65%) together with 23 ml of DV20 were added. The PP container was sealed with parafilm and put on an orbital shaker (˜ 60 rpm). The reaction continued for 16 h at room temperature. Afterwards, the discs were washed with 150 ml DV20×6×20 min. Titration gave an ionic capacity of 14 μmol/ml.

A deactivation solution was prepared: Ethylenediaminetetraacetic acid, disodium dihydrate (EDTA*Na2*2H2O, 61 mg) and di-Sodium hydrogen phosphate dodecahydrate (Na₂HPO₄*12H₂O, 5.7 g) were added to deionized water (150 ml). After 5 minutes stirring thioglycerol (12 ml) was added and the pH adjusted to 8.3.

12 discs of Fibro-VS were suspended in the above deactivation solution and gently stirred for 16 hours at room temperature. Thereafter, the discs were washed with DV20 3 times, once with 1 M NaCl and 3 times with DV20. Each wash was performed with 150 ml of solution with a contact time of 20 minutes.

### Vinylsulfone Deactivation Before DEAE Coupling

The deactivation was performed following the procedure described above. Thereafter, 12 discs were added together with 150 ml DV20 and 32 g NaSO₄ in a 600 ml beaker at 300 C, followed by 13 g KOH. The temperature was raised to 30° C. and 2-(Diethylamino) ethylchloride hydrochloride (65% solution) was added respectively 6, 12, 15 and 19 ml for the different prototypes. The reaction was proceeded for 19 hours. Thereafter the reaction was neutralized to pH˜7 using 1M HCl solution. The prototypes were then washed with 300 ml DV20 for 8 times (20-30 min) in the beaker setup. Titration gave respectively an ionic capacity of 122, 193, 207 and 244 μmol/ml

### N,N-Diethylethylenediamine Functionalization of Fibro (DAX (DiAmino Exchange) Fibro) (FIG. 3b)

The Fibro material was functionalized as illustrated in FIG. 4b. Fibers with vinylsulfone reactive groups are reacted with N,N-diethylethylenediamine to form a functionalized matrix with N,N-diethylethylenediamine groups, forming a Fibro DAX (DiAmino exchange) material. The following protocol was used for the functionalization: N,N-Diethylethylenediamine coupling solutions were formed:
1) 1.5% in water
2) 3% in water

20 discs Fibro-VS were placed in container together with 25 ml of the respective coupling solution. The reaction was left for 16 hours. After this time prototypes were washed with 150 mL DI water, place back on orbital shaker for ˜ 20 mins. Repeat the water washing process 5 times. Titration gave respectively an ionic capacity of 169 and 223 μmol/ml.

deactivation was performed following the procedure described above for Fibro DEAE

### N,N-Dimethylethylenediamine (DMEN) Functionalization of Fibro (FIG. 3d)

20 discs of washed Fibro-VS sheet were placed in a container. A solution of 3 v/v % N,N-Dimethylethylenediamine (DMEN ligand) in milli-Q water (740 μl DMEN ligand in 24.26 ml DV20) was added to the container and placed on an orbital shaker. The reaction was let for 19 hours at room temperature. Thereafter, the supernatant was discarded and replaced with 150 mL DI water, place back on orbital shaker for 20 mins. Repeat the water washing process 5 times. Finally, the deactivation was performed following the procedure described above. Titration gave an ionic capacity of 230 μmol/ml.

Functionalization of Fibro with N,N-Dimethylethylamine (DMAE or DMEA) (FIG. 3c)

The following protocol was used for the functionalization:

20 discs of washed Fibro-VS sheet were deactivated according to the procedure described in above. A 65% solution of 2-Chloro-N,N-dimethylethylamine hydrochloride (DMEA) (16.252 g)) was prepared by dissolving it in DV20 (8.750 g) together with Na2SO4 (31.96 g aqueous 1.5 M). It was then added in a beaker containing 150 ml DV20 and KOH (12.69 g, 1.5 M, about pH 13.3). The reagent solution was then added to the 20 deactivated discs in a container, which is placed on an orbital shaker at room temperature. The reaction continued for 19 h under shaking. The solution was later neutralized to pH 7 using 1:1 HCl: DV20 solution. The prototype was then washed with DV20 3 times, with 1 M NaCl 3 times and finally with DV20 2 times. Each wash was for approximately 20 min. Titration gave an ionic capacity of 52 μmol/ml.

The formed functionalized support materials, hereinafter called AIEX (anion exchange) Fibro material, were attached into membrane supports. The membrane diameter was 23 mm and the membrane volume ˜0.35 mL.

### Magnetite Agarose Base Beads

Magnetite Agarose Base Beads (Mag) Functionalized with DEAE (Mag DEAE)

10 g of MagSepharose 4FF (1×10 GV of DV20) was washed with 10 volume of DV20, drained filtered and added into a Falcon tube together with 3 ml DV20 and 1.8 g (12.7 mmol) sodium sulphate. The tube was put into a shaking table in 45 minutes. Thereafter, 2.2 ml NaOH 50% were added and the tube was shaken for an extra 10 minutes. 1 ml of 2-Chloro-N,N-diethylethylamine was then added and the reaction was left at 30° C. on a shaking device with a rotation of 600 rpm for 17 hours. Thereafter, the resin was washed with 6×1GV DV20, 3×1GV 2M NaCl and 3×1GV DV20. The gel was titrated for Cl concentration resulting in ion capacity of 34 μmol/ml of resin. Following the same procedure as above but using instead 5 ml of 2-Chloro-N, N-diethylethylamine resulted in a resin with an ionic capacity of 93 μmol/ml of resin.

Magnetite Agarose Base Beads (Mag) Functionalized with DEAE Dextran T40 (Mag DEAE Dextran) (FIG. 4c)

50 g of MagSepharose 4FF was washed with 10×1 GV of DV20, drained and transferred to a 250 ml three-necked round-bottle together with 17 ml DV20 and put in a 27° C. water bath with overhead stirrer ˜300 rpm. After two minutes, 5.5 g (0,138 mole) NaOH pellets were added to the slurry. After 15 minutes, 21.3 ml (0.23 mole) of epichlorohydrin was added and the reaction was left for 2 hours. The resin was then washed with DV20 until the washings reach neutral pH.

45 g of epoxy activated MagSepharose was transferred to a 250 ml three-necked round-bottle containing a dextran T40 solution (20.5 g, 13 mL deionized water). The reaction was placed at 40° C. with overhead stirrer ˜120 rpm. 1.5 ml of deionized water was added and after 20 minutes of stirring, nitrogen gas bubbles were used in the solution to drive away oxygen bubbles. Thereafter, 2.5 ml of 50% NaOH and 100 mg sodium borohydride (NaBH4) was added to the system and the reaction underwent 18 hours.

The prototype was synthesised in the same way as described for DEAE MagSepharose The gel was titrated for Cl concentration resulting in ion capacity of 143 μmol/ml of resin

### Anion Exchange Chromatography

### Fibro Support Material

The above-described functionalized support materials were used as anion exchange chromatography media in the following anion exchange chromatography experiments when purifying lentivirus particles. In table 1 is listed all AIEX Fibro support materials used in the experiments.

TABLE 1
AIEX Fibro prototypes

| | Ligand |
|---|---|
| | density |
| Functionality | (μmol/ml) |
| Fibro DEAE | 14 |
| Fibro DEAE | 122 |
| Fibro DEAE | 193 |
| Fibro DEAE | 207 |
| Fibro DEAE | 244 |
| Fibro DAX | 169 |
| Fibro DAX | 223 |
| Fibro DMEN | 230 |
| Fibro DMAE | 52 |

### Elution Buffer

All AIEX Fibro prototypes in table 1 were first tested in a step-gradient with increasing salt concentrations in the elution buffer at a flow of 10 ml/min. A 10 ml LV feed was applied on the columns in all cases. The step gradient is described in table 2 below. A-buffer (running buffer) was 20 mM TRIS pH 7.4 and B-buffer (elution buffer) was 20 mM TRIS pH 7.4 with increasing NaCl concentrations in the five elution steps. A major part of the 10 lentivirus particles was shown to be eluted using a salt concentration of 0.65 M or lower.

TABLE 2
Step-gradient for initial evaluation
of AIEX prototypes

| | Salt | |
|---|---|---|
| Elution | Concentration | Fraction |
| step | (NaCl) | volume |
| 1 | 0.2M | 10 mL |
| 2 | 0.45M | 10 mL |
| 3 | 0.65M | 10 mL |
| 4 | 1.0M | 10 mL |
| 5 | 1.3M | 10 mL |

A CaptoCore700 column: 1 mL Cytiva Capto™Core 700 multimodal chromatography resin was packed into a 1 mL Tricorn 5 column. The CaptoCore column was applied in-line after the anion exchanger to absorb possible residual impurities.

From the DEAE prototypes with different ligand densities/ionic binding capacities (ICs) Fibro DEAE with an IC of 244 μmol/ml showed the best overall Lentivirus yield, see table 4 and 5 below. The prototypes with lower ICs than 244 μmol/ml showed lower Lentivirus yields (VP %). However, Fibro DEAE IC 193 μmol/ml was chosen as the one with overall best performance since the Lentivirus elutes at lower salt concentrations. For Fibro DEAE IC 193 μmol/ml the highest amount of Lentivirus elutes at 0.45 M NaCl instead of 0.65 M NaCl as for Fibro DEAE IC 244 μmol/ml. For Fibro DEAE IC 193 μmol/ml the CTQ (critical to quality) is elution at salt concentrations <500 mM, thereby allowing recovery of more viable virus particles. Furthermore, the eluate of Fibro DEAE IC 193 μmol/ml contains less impurities, especially host DNA (total DNA (%) in table 4 and 5). In table 4 and 5 hcp (host cell protein) levels are also shown. ND (not detected) in the tables indicate that the levels of for example hcp were below the detection limit.

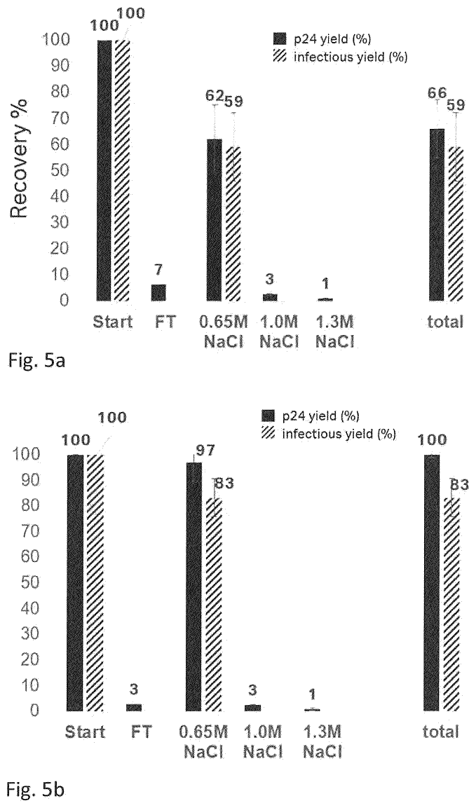

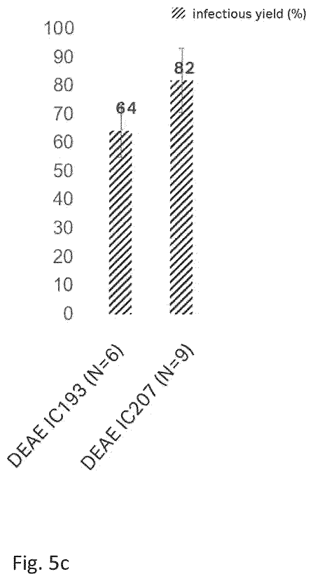

In FIGS. 5a-5c is a comparison of lentivirus capturing using Fibro DEAE IC193 and Fibro DEAE IC207, respectively, as the anion exchange chromatography medium, using step elution with increasing salt concentration. (Striped pattern show infections recovery and filled black bars show total virus particles (p24 ELISA)). FIG. 5c shows the reproducibility of lentivirus capturing using the anion exchange chromatography materials of FIG. 5a and FIG. 5b, respectively. These results indicate that using Fibro DEAE IC207, there might be a higher total recovery and higher elution at lower salt concentration than using Fibro DEAE IC193 as the anion exchange chromatography medium.

TABLE 3
Fibro DEAE with IC 14 and 122 μmol/mL membrane.

| | Fibro DEAE IC 14 | | | Fibro DEAE IC 122 | | |
|---|---|---|---|---|---|---|
| | Remaining Yield (%) | | | Remaining Yield (%) | | |
| | Lentivirus | HCP | total DNA | Lentivirus | HCP | total DNA |
| | (VP %) | (%) | (%) | (VP %) | (%) | (%) |
| Flow-Through | 7 | 70 | 72 | 1 | 30 | 48 |
| Eluate 1: 0.2M | 6 | ND | ND | ND | ND | ND |
| NaCl | | | | | | |
| Eluate 2: 0.45M | 2 | ND | ND | 7 | ND | 33 |
| NaCl | | | | | | |
| Eluate 3: 0.65M | 1 | ND | ND | 1 | ND | ND |
| NaCl | | | | | | |
| Eluate 4: 1M | 1 | ND | ND | ND | ND | ND |
| NaCl | | | | | | |
| Eluate 5: 1.3M | ND | ND | ND | ND | ND | ND |
| NaCl | | | | | | |
| Total Elution | 9 | ND | ND | 8 | ND | 33 |
| Yield | | | | | | |

TABLE 4
Fibro DEAE with IC 193 and 244 μmol/ml

| | Fibro DEAE IC 193 | | | Fibro DEAE IC 244 | | |
|---|---|---|---|---|---|---|
| | Remaining Yield (%) | | | Remaining Yield (%) | | |
| | Lentivirus | HCP | total DNA | Lentivirus | HCP | total DNA |
| | (VP %) | (%) | (%) | (VP %) | (%) | (%) |
| Flow-Through | ND | 63 | 45 | 5 | 67 | 41 |
| Eluate 1: 0.2M | 1 | 1 | ND | 3 | 1 | 12 |
| NaCl | | | | | | |
| Eluate 2: 0.45M | 11 | 1 | 25 | 4 | ND | 23 |
| NaCl | | | | | | |
| Eluate 3: 0.65M | 9 | ND | 1 | 21 | ND | 15 |
| NaCl | | | | | | |
| Eluate 4: 1M | 2 | ND | ND | 4 | ND | 11 |
| NaCl | | | | | | |
| Eluate 5: 1.3M | ND | ND | ND | 1 | ND | 10 |
| NaCl | | | | | | |
| Total Elution | 23 | 2 | 27 | 34 | 1 | 70 |
| Yield | | | | | | |

Of the two DAX prototypes, Fibro DAX IC 223 μmol/ml showed the highest Lentivirus elution yield of all of the tested prototypes (see table 6 below). However, the Lentivirus viability was 0 for DAX IC 223 μmol/ml, which will be investigated further.

TABLE 5
Fibro DAX prototypes

| | Fibro DAX IC 169 | | | Fibro DAX IC 223 | | |
|---|---|---|---|---|---|---|
| | Remaining Yield (%) | | | Remaining Yield (%) | | |
| | Lentivirus | HCP | total DNA | Lentivirus | | total DNA |
| | (VP %) | (%) | (%) | (VP %) | HCP (%) | (%) |
| Flow-Through | 1 | 70 | 53 | 1 | >99 (199) | >99 (186) |
| Eluate 1: 0.2M | 1 | 1 | 12 | 4 | 1 | 39 |
| NaCl | | | | | | |
| Eluate 2: 0.45M | 1 | 1 | 13 | 56 | 1 | 14 |
| NaCl | | | | | | |
| Eluate 3: 0.65M | 20 | ND | ND | 15 | ND | 13 |
| NaCl | | | | | | |
| Eluate 4: 1M | 7 | ND | ND | 6 | ND | 10 |
| NaCl | | | | | | |
| Eluate 5: 1.3M | 2 | ND | ND | 3 | ND | ND |
| NaCl | | | | | | |
| total elution | 31 | 2 | 25 | 83 | 1 | 76 |
| yield | | | | | | |

Fibro DMEN showed low Lentivirus yield as well as a high amount of DNA impurities. Fibro DMAE had the highest amount of Lentivirus eluting at 0.2 M NaCl (see table 7), however the overall yield was lower than for DAX IC 223 μmol/ml.

TABLE 6
Fibro DMEN and DMAE prototypes

| | Fibro DMEN | | | Fibro DMAE | | |
|---|---|---|---|---|---|---|
| | Remaining Yield (%) | | | Remaining Yield (%) | | |
| | Lentivirus | HCP | total DNA | Lentivirus | HCP | total DNA |
| | (VP %) | (%) | (%) | (VP %) | (%) | (%) |
| Flow-Through | ND | 79 | 48 | ND | 61 | 77 |
| Eluate 1: 0.2M | 2 | ND | 14 | 16 | ND | 19 |
| NaCl | | | | | | |
| Eluate 2: 0.45M | 1 | ND | 30 | 10 | ND | 10 |
| NaCl | | | | | | |
| Eluate 3: 0.65M | 8 | ND | 13 | 2 | ND | 7 |
| NaCl | | | | | | |
| Eluate 4: 1M | 6 | ND | 8 | 2 | ND | 7 |
| NaCl | | | | | | |
| Eluate 5: 1.3M | 1 | ND | 7 | 1 | ND | 7 |
| NaCl | | | | | | |
| total elution | 18 | ND | 72 | 31 | ND | 50 |
| yield | | | | | | |

### Mag Support Material

The support materials Mag DEAE IC 34 μmol/ml, Mag DEAE IC 93 μmol/ml and Mag DEAE Dextran IC 143 μmol/ml, were evaluated in the attempt to bind and elute Lentivirus.

10% slurries of the different Mag support materials were produced. ˜1.1 mL of a Mag support material was poured into the 1.0 mL “Cube” and vacuum suction was applied. The 1 mL gel plug was transferred with milli-Q water into a 50 mL falcon tube with ˜10 mL binding buffer. The resin was washed with 3×10 mL binding buffer and the beads were trapped on a magnet between washing steps. In the last washing step the beads were trapped on a magnet and all excess liquid was removed using a pipet. Finally, 9.0 mL binding buffer was pipetted into the resin to obtain a 10% resin slurry.

The binding capacity and elution yield of the three different Mag support materials were investigated. 10, 25, 35 and 50 μL of each support material was pipetted into two the deep well plates. The beads were incubated with 0.5 mL lenti sample, washed with binding buffer and eluted. The titer of lenti in the samples after 1 h incubation and the pooled eluates were determined using the p24 ELISA. Total protein and DNA was also determined for the start sample and the eluates. Impurity levels in the eluates using 1.3 M NaCl was investigated. DNA reduction is a challenge while the total protein were reduced in the order of 1-2 logs.

The three support materials were evaluated with elution buffer with different salt content to estimate at which conductivity the lentivirus elutes. Lenti bound to the prototypes were eluted at 0.4, 0.65 and 1.3 M NaCl in 20 mM Tris pH 7.4. In this study 50 μl resin was used and 500 μl lenti sample.

The binding capacity for the different support materials incubated with 500 μL Lentivirus sample were investigated. All materials showed binding to LV. The prototype with dextran and DEAE attached onto the resin showed higher capacity than the two other support materials.

Looking at elution yield all materials showed >30% elution yield of LV using 1.3 M NaCl in the elution buffer. The Mag DEAE Dextran material showed also promising LV elution yield, >70%.

All support materials bind Lenti virus and Mag DEAE Dextran IC 143 μmol/ml showed highest binding capacity, ˜4e10 capsids/mL resin. It was possible to elute Lenti from all support materials using 0.4 M NaCl in the elution buffer but with different elution yields. Generally, highest elution yield was obtained using 0.65 M NaCl and the DEAE material with low ligand density showed highest elution yield (>65% at 0.4 M NaCl) but the binding capacity was 10 fold lower than for the Mag DEAE Dextran material that showed an elution yield of 23% at 0.4 M NaCl.

### Discussion

Other support materials than the functionalized Fibro materials and functionalized magnetite agarose beads described above may be used as support materials. Examples of such support materials are functionalized membranes, monoliths, porous beads, non-porous beads, and expanded bed media.

Such functionalized support materials will also work as anion exchange chromatography medium in purification of enveloped virus particles or exosomes as long as the support material allow the enveloped virus particle or exosome to be in contact with the ligands. The type of support (porosity, synthetic beads, etc.), however, will have a strong impact on the performance.

Some binding of enveloped virus particles or exosomes will happen within the specified ionic capacity range of 10-500 μmol/mL, since it is an electrostatic interaction. An optimum ionic capacity value is connected with the need to have enough ligand to ensure binding but not too high ionic capacity such that the binding is too hard and cause a decrease in recovery. It is likely possible that the optimum ionic capacity is strongly dependent on the support material used (ligand density by surface of contact). For fibro the optimum IC seems to be around 100-250 μmol/mL).

The results above are shown for the enveloped virus Lentivirus. Similar results are obtainable also with other enveloped virus types, such as DNA viruses and RNA viruses and exosomes.

1. An anion exchange chromatography medium for use in purification of enveloped virus particles or exosomes from a feed, the anion exchange chromatography medium comprising a support material being functionalized with a ligand comprising a diamine functionality generating at least one weak anion exchange group to an ionic capacity of 10-500 μmol/mL.

2. The anion exchange chromatography medium of claim 1, wherein the weak anion exchange group is positively charged or partially positively charged at a pH of 6-10.

3. The anion exchange chromatography medium of claim 1, wherein the ligand or a portion of the ligand is described by the formula: 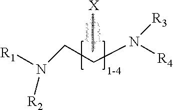
wherein X is selected from H, OH or a C₁₋₃ group, and
R1, R2, R3 and R4 are independently selected from H, and a C₁₋₃ group,
wherein a C₃ group is straight or branched,
wherein a C₁₋₃ group comprises groups independently selected from OH, O—C₁₋₂, S—C₁₋₂, NH, NHR, NR₂,
wherein R is selected from H and a C₁₋₃ group.

4. The anion exchange chromatography medium of claim 3, wherein the ligand is selected from N,N,N′-triethylethylenediamine, diethylenetriamine, N,N′-dimethylethylenediamine, N-methylethylenediamine, 1,3-diaminopropane, 1,3-diamino-2-hydroxypropane, 2-methyl-1,3-propanediamine and N,N-diethylethylenediamine.

5. The anion exchange chromatography medium of claim 1, wherein the ligand is N,N-diethylethylenediamine.

6. The anion exchange chromatography medium of claim 1, wherein the support material is selected from monoliths, membranes, porous beads, non-porous beads, magnetic beads, or expanded bed media.

7. The anion chromatography medium of claim 1, wherein the support material is a non-woven fibrous material having an effective pore size of 0.1-2.0 μm.

8. The anion exchange chromatography medium of claim 1, wherein the ligand is connected to the support material through an extender group selected from polysaccharide structures and polymeric structures.

9. An anion exchange chromatography comprising the anion exchange chromatography medium of claim 1.

10. A process of purifying enveloped virus particles or exosomes from a feed, the process comprising:
obtaining a solution comprising the enveloped virus particles or exosomes and one or more impurities,
adding the solution to the anion exchange chromatography medium of claim 1 at a pH of 6-10,
eluting the encapsulated virus particles or exosome from the anion exchange chromatography medium by contacting the anion exchange chromatography medium with an elution buffer having a salt concentration of at most 0.65 M, collecting the thus formed eluate containing enveloped virus particles or exosomes.

11. The process of claim 10, further comprising after the eluting step, adding the eluate to a multimodal chromatography resin.
